(19) United States
(12) Patent Application Publication
(10) Pub. No.: US 2026/0241083 A1
(43) Pub. Date: Aug. 20, 2026

(54) Breast pump

(71) Applicant: MEDELA AG, Baar (CH)

(72) Inventors: Charles Kim, North Chicago, IL (US); Kalpesh Patel, Woodstock, IL (US)

(21) Appl. No.: 19/057,750

(22) Filed: Feb. 19, 2025

(51) Int. Cl. A61M 1/06

(52) U.S. Cl. CPC A61M 1/0693; A61M 1/062; A61M 2202/0071; A61M 2205/3351; A61M 2210/1007

(57) ABSTRACT

A breast pump including a breast shield adapted to at least in part receive a breast of a lactating mother or user. The breast pump has an aggregate for generating a cyclic suction profile for expressing milk from the breast. The breast pump has a milk container providing a reservoir for the expressed milk and a controller for controlling the aggregate. The aggregate may be formed by a motor and a pump transferring the rotational movement of the motor into at least one cyclic suction profile, which suction profile includes a vacuum start phase, a vacuum application phase, a vacuum maintenance phase and a vacuum release phase consecutive to each other and specifically designed with respect to relative duration within the suction cycle and pressure effective in respective phase to optimize the vacuum application to the breast of the user with the scope to obtain an increase milk extraction.

### FIELD OF THE INVENTION

The present invention relates generally to a breast pump. Specifically, the present invention relates to a breast pump including a breast shield adapted to at least in part receive a breast of a lactating mother or user. The breast pump furthermore has an aggregate for generating a cyclic suction profile for expressing milk from the breast. The breast pump has a milk container providing a reservoir for the expressed milk and a controller for controlling the aggregate. The breast shield usually has or is in communication with a nipple tunnel providing a fluid passage for the expressed milk from the breast towards the reservoir. The aggregate may be formed by a motor and a pump transferring the movement of the motor into at least one cyclic suction profile or vacuum curve.

### BACKGROUND

Breast pumps for use by nursing mothers are well known. They allow the nursing users to express the breastmilk as necessary or convenient, and further provide collection of the breastmilk for later use. For some users, breast pumps may be a necessity, such as when the child has suckling problems, or if the user has problems with excessive or deficient milk production, or soreness, deformation, or injury of the mammilla. Of particular instance herein is the situation confronting a user where the infant is premature, and therefore is most always separated from the mother.

Electrically-driven breast pumps are commonplace, typically including a vacuum pump which has an electric motor that plugs into standard house current, and/or operates with battery power. Advantages of this type of pump are convenience, ready controllability and regulation of the vacuum, and in many instances the ability to pump both breasts at once.

Electrically-driven, motorized breast pumps generally have a driving mechanism for generating the vacuum (negative pressure) to be applied at the breast geared to a particular sequence, or curve, of vacuum or negative pressure increase (i.e. increasing suction), and then release. The cycling vacuum reproduces baby behavior and has at least a stimulation pattern and an expression pattern; examples of such patterns are reproduced in WO 2021 160 875, WO 2010 096 547, WO 2020 178 737, WO 2008 034 193, WO 2022 132 565, WO 02 053 208 and WO 03 082 378. Each pattern is made of vacuum curves that are cyclically reproduced by the vacuum pump.

Among those WO 2010 096 547 discloses a pumping curve with fast vacuum application.

WO 2020 178 737 also teaches a pumping curve with vacuum pulsation.

WO 2008 034 193 teaches pumping curves with fast vacuum application.

WO 02 053 208 teaches a pumping curve with improved vacuum application.

Finally, WO 2022 132 565 teaches a pumping curve with pulsating fast vacuum application.

### UNDERLYING PROBLEM

The pumping curves known from the prior art do not allow optimization of the vacuum application to the breast and thus milk extraction from the breast. For this, the above prior art teaches to maintain as much vacuum as possible and generate the maximum suction/negative pressure as fast as possible.

### SUMMARY OF THE INVENTION

The present invention proposes an optimized vacuum application to the breast of a lactating user, in which the vacuum application is optimized with the scope to obtain an increase milk extraction.

It was discovered that in order to optimize vacuum application different aspects have to be taken into account.

It was in particular discovered that four phases can be identified in a pumping curve that if properly set up allow an optimal vacuum application to the breast and maximization of the time and level of vacuum that is applied to the breast.

A first phase of the suctions cycle, which is identified as a vacuum start phase of this suctions cycle gently applies vacuum to the breast, so that the breast can set in the breast shield and allows a fast and deep vacuum application occurring subsequently; this part is typically not useful for milk expression but is only relevant for comfort and breast preparation to the vacuum application.

A second phase of the cycle, which is identified as a vacuum application phase, applies vacuum in a fast way in order to start and maintain milk expression. The slope is fast in order to ensure fast vacuum application and thus efficient milk extraction.

A third phase, which is identified as a vacuum maintenance phase, maintains the vacuum substantially constant, this is useful to increase vacuum application and thus milk extraction.

A fourth phase, which is identified as a vacuum release phase, is less relevant for optimized milk extraction. The vacuum release phase is provided to increase the pressure and thereby reduce the vacuum to raise the pressure to the initial pressure at the beginning of the vacuum start phase. The vacuum release phase may be rather short. The duration thereof may be dominated by the inertia of the breast pump, in particular the limited capability of the breast pump with the breast shield sealed against the breast of the user to allow air to enter into the nipple tunnel of the breast shield and thus reduce the pressure for the nipple and therefore the suction pressure driving the expression of milk from the breast of the user.

As far as the present description refers to vacuum levels, it refers to the pressure within the nipple tunnel as measurable by a sensor. This does not mean that the breast pump of the present invention needs to have such a sensor. A sensor may e.g. be used to calibrate the type of the breast pump in order to store measured pressure values along with operational parameters of the pump in order to reproduce the pressure performance by applying respective operation parameters without utilizing a sensor for monitoring or controlling the resulting pressure condition. Thus, the pressure condition referred to in this specification may correspond with an actual pressure condition within the nipple tunnel and expected to be created for exposing the breast of the user to such pressure condition by controlling specific operational parameters and assuming that the breast pump will reproduce the pressure conditions assigned to those operational parameters of the pump.

### DESCRIPTION OF THE INVENTION

The breast pump has a breast shield connected to the aggregate possibly powered by a built in battery or connection to the power via a cable. The connection between the breast shield and the pump can occur via a tube but also in-bra solutions are possible, in which the breast shield, the housing including the aggregate and the control and possibly the battery, as well as the milk container form a unitary body, which body is supported and held in place by the bra of the user.

The pump includes an aggregate for generating vacuum that is connected to the breast shield. The aggregate may comprise a motor like an electric motor and a piston e.g. a cylinder/plunger and is of e.g. multi stroke type. Thus, more than one run of the plunger is required to reach the maximum vacuum. In other words, for each run of the plunger the aggregate generates a vacuum step.

Advantageously, the pulsation is achieved via the multi stroke aggregate, so that at each run of the plunger the aggregate generates and transmits to the breast shield one vacuum step.

After the plunger has completed its run and moves back to its starting position to prepare for a subsequent run, vacuum can be slowly released, so that pulsation may be achieved.

The pump usually has a selector to select the maximum level of vacuum that is applied to the breast.

Typically, the user who uses the breast pump selects the maximum vacuum that she can withstand and the pump applies vacuum to the breast according to vacuum curves having the selected maximum vacuum.

The controller is usually adapted to provide multiple vacuum curves, e.g. often 10 or more, e.g. 16. These vacuum curves are defined by a same base curve that is scaled according to different levels of maximum vacuum. For a diagram with time on the horizontal axis and vacuum of the vertical axis, scaling implies application of the same reduction coefficient on both axes.

It is important that the user selects the curve with the highest level of vacuum, because this way the applied vacuum is maximized, and it is also important that this curve is maintained for the whole of the pumping cycle.

The curves according to the invention have four phases, each with a specific scope, in order to
maximize the vacuum that is applied with that specific curve, i.e. for each curve the area below the curve is optimized/maximized, so that the curve with that specific maximum vacuum maximizes the area below the curve; the area below the curve is proportional to the extracted milk.
allow the user to select and use (for the whole of the pumping cycle) the curve with the highest vacuum level possible, according to her sensibility, among the vacuum levels that are available from the breast pump.

In the breast pump of the present invention the controller is adopted to control the aggregate to provide at least one cyclic suction profile consisting of a vacuum start phase, a vacuum application phase, a vacuum maintenance phase and a vacuum release phase, wherein the vacuum start phase is followed by the vacuum application phase, wherein the vacuum application phase is followed by the vacuum maintenance phase, wherein the vacuum maintenance phase is followed by the vacuum release phase, wherein the vacuum release phase defines the end of the cycle and is followed by the vacuum start phase of the next cycle.

The four phases mentioned above immediately follow each other with no other phase then the four phases mentioned above contained within the cyclic suction profile all cycle. Accordingly, the cycle starts with the vacuum start phase and terminates with the vacuum release phase, which vacuum release phase is followed by the vacuum start phase of the next cycle.

In the following, the characteristics of the different phases of the breast pump of the present invention will be described. Negative pressure gradients indicate a phase of increasing suction force within the nipple tunnel. The gradients are identified by mmHg/per second. The duration of each phase is identified in percent relative to the total duration of the cycle. As each cycle consists of the four phases, the total duration of the four phases will sum up to 100%.

1. Vacuum Start Phase

Here the vacuum is applied in a gentle way; this prepares the breast to the subsequent fast application and long maintenance of the vacuum, so that the user only sentence is also able to withstand high vacuum levels, i.e. she can withstand and select a pumping curve with higher vacuum level.

The vacuum start phase is defined by:
Duration of this phase is between 15-21% of the cycle and preferably 18% with some tolerance around his value.
Slope of this phase is −80 to-120 mmHg/s and preferably-100 mmHg/s with some tolerance around his value.

This vacuum start phase can preferably have a first subphase and a second subphase defined as follows.

### First Subphase

Duration of this phase is between 7-13% of the cycle and preferably 10% with some tolerance around his value.
Slope of this phase is −15 to-25 mmHg/s and preferably −20 mmHg/s with some tolerance around his value.

### Second Subphase

Duration of this phase is between 5-15% of the cycle and preferably 10% with some tolerance around his value.
Slope of this phase is −195 to-250 mmHg/s and preferably-200 mmHg/s with some tolerance around his value.

### 2. The Vacuum Application Phase

This phase applies the vacuum in a fast way; the user can withstand it because the breast is properly set in the breast shield and is already prepared for the vacuum application.

The vacuum application phase is defined by:
Duration of this phase is between 25-35% of the cycle and preferably 30% with some tolerance around his value.
Average slope of this phase is −600 to-700 mmHg/s and preferably −650 mmHg/s with some tolerance around his value.
This vacuum application phase has pulsed vacuum application, with maximum vacuum steps of between −18 and −25 mmHg and preferably −20 mmHg with some tolerance around his value. While the control may order an idealized stepped configuration in which strictly vertical lines indicating instantaneous reduction of the pressure by the step value of e.g. 20 mmHg followed by a strictly horizontal lines indicating constant pressure over time until the next step is ordered, the actual slope of the vacuum application phase may exhibit some expiration of time due to inertia as the step is applied and may exhibit some increase in the pressure until the next step is ordered due to leakage.

### 3. Vacuum Maintenance Phase

Vacuum maintenance phase allows maximization of the vacuum applied by the specific curve.

The vacuum maintenance phase is defined by:
Duration of this phase is between 23-33% of the cycle and preferably 28% with some tolerance around his value.
Slope of this phase is −10 mmHg/s and 10 mmHg/s and preferably −3 mmHg/s with some tolerance around his value. Thus, in the vacuum maintenance phase the vacuum is substantially constant but may slightly increase or it can also decrease. The control is usually controlled to keep the pressure in vacuum maintenance phase constant. However, some leakage may lead to a slight increase of the suction pressure i.e. higher pressure values. Adjustment to compensate for such leakage may lead to a further and slight decrease of the pressure. The other phases may exhibit at least predominately a generally continuous slope.

### 4. Vacuum Release Phase

In this phase vacuum is released.

The vacuum release phase is defined by:
Duration of this phase is between 17-27% of the cycle and preferably 22%
Slope of this phase is not important and may be fast, e.g. 1000 mmHg/s or more. The vacuum release phase may comprise a maximum slope of between 900 and 1500 mmHg/s. As mentioned before, the controller could be adopted to allow instantaneous relaxation of the negative pressure from the pressure value during the vacuum maintenance phase to a baseline pressure around 0 mmHg or slightly below this value, while the actual pressure condition in the nipple tunnel may more slowly adopt due to inertia of the system, in particular the reduced capability to allow ambient air to quickly enter into the nipple tunnel.

### DESCRIPTION OF THE DRAWINGS

These and other advantages of the invention will be further understood upon consideration of the following detailed description, in which:

FIG. 1 is a perspective view of an embodiment of a breast pump and

FIG. 2 is a schematic graph of different phases of a cyclic suction profile controlled by the breast pump of the present invention.

### DETAILED DESCRIPTION OF THE DRAWINGS

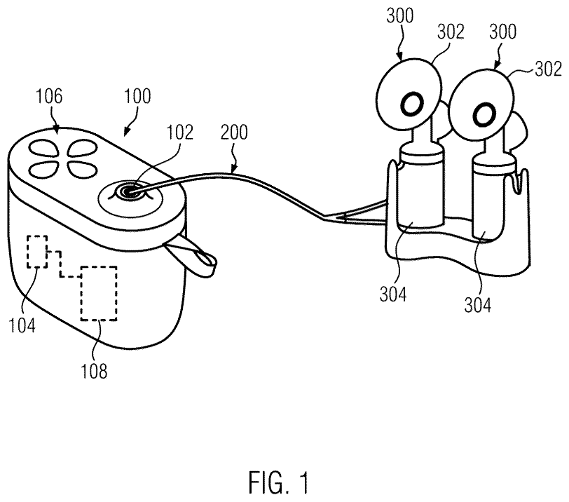

FIG. 1 shows a breast pump 100 with a drive interface 102 and a controller 104 operatively connected to a controller interface 106. The controller 104 controls an aggregate 108 for providing a cyclic suction profile at the drive interface 102. Via a tube 200 the drive interface 102 is connected to breast shield components 300 each having a breast shield 302 adapted to receive at least in part the breast of a lactating user and a reservoir 304 for receiving the expressed milk. The cyclic suction profile of the breast pump 100 will provide cyclic suction within the breast shield 302 for expressing milk from the breast of the user when using the breast pump 100.

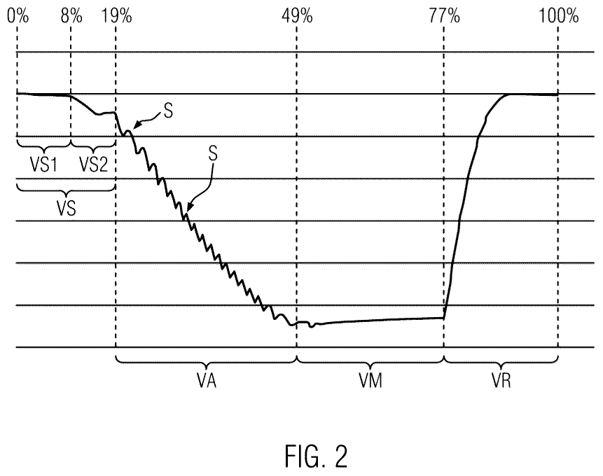

FIG. 2 shows a graph with a vacuum curve, which may be one of a set of curves available to be chosen in such a breast pump or any kind of breast pump. Each vacuum curve has a vacuum start phase VS, a vacuum application phase VA, a vacuum maintenance phase VM and a vacuum release phase VR. Those four phases are consecutive phases and exclusively define a cyclic suction profile, which cyclic suction profile starts at time 0 seconds (time at the abscissa in FIG. 2) at the beginning of the vacuum start phase VS and terminates with 1.19 seconds at the end of the vacuum release phase VR.

The vacuum start phase VS is divided into two subphases. The vacuum start phase VS separate consists of a first subphase VS1 and a second subphase VS2 following the first subphase VSU1. In the first subphase VS1 the suction pressure is almost constant and at most slightly reduced with an average gradient or slope of not more than 25 mmHg (absolute value). In the second subphase VS2 the suction pressure is reduced with a more pronounced gradient or slope of about −200 mmHg.

In the second phase, i.e. the vacuum application phase VA, the negative pressure is reduced in steps S of about −20 mmHg. The pressure curve of FIG. 2 shows a slight increase in pressure after each step S due to some degree of leakage. In other words, after each step S the pressure is slightly raised before the next step S is ordered bringing the suction pressure to a new minimum value of the pressure effective in the nipple tunnel.

In the third phase, i.e. the vacuum maintenance phase VM, the negative pressure is almost constant. In fact, FIG. 2 shows a slight increase of the negative pressure during the vacuum maintenance phase VM.

This vacuum maintenance phase VM is followed by the vacuum release phase VR, in which the negative pressure applied during the vacuum maintenance phase VM is released to assume the initial pressure of the vacuum start phase VS, which vacuum start phase VS is the first phase of the next cycle, which cycle shows the same phases as discussed before with the same characteristics of each phase.

In the embodiment of FIG. 2, the vacuum application phase VA is the longest phase of the cycle whereas the vacuum start phase VS is the shortest phase of the cycle. The vacuum release phase VR has a longer duration than the vacuum start phase VS. The vacuum maintenance phase VM has a longer duration than the vacuum release phase VR but a shorter duration in comparison to the vacuum application phase VA.

The above description and details elucidate the inventors findings according to which an optimized vacuum application to the breast of a lactating user will increase milk extraction. It was in particular discovered that the four phases identified above in a properly set pumping curve/pumping cycle will allow an optimal vacuum application to the breast and maximization of the time and level of vacuum that is applied to the breast.

### LIST OF REFERENCES

100 breast pump
102 drive interface
104 controller interface
106 controller
108 aggregate
200 tube
300 breast shield components
302 breast shield
304 reservoir
VM vacuum maintenance phase
VR vacuum release phase
VA first application phase
VS vacuum start phase
VS1 first subphase of vacuum start phase
VS2 second subphase of vacuum start phase

What is claimed is:

1. A suction profile for a breast pump comprising a vacuum start phase, a vacuum application phase, a vacuum maintenance phase and a vacuum release phase, wherein the vacuum start phase is followed by the vacuum application phase, wherein the vacuum application phase is followed by the vacuum maintenance phase, wherein the vacuum maintenance phase is followed by the vacuum release phase, wherein the vacuum release phase defines the end of the cycle,
wherein the vacuum start phase extends over between 15 to 21% of the cycle duration and has and has an average pressure slope of between −80 and −120 mmHg/s,
wherein the vacuum application phase extends over between 25 to 35 % of the cycle duration and has an average pressure slope of between −600 and −700 mmHg/s,
wherein the vacuum maintenance phase extends over between 23 to 33 % of the cycle duration and has an average pressure slope of between −10 mmHg/s and 10 mmHg/s,
wherein the vacuum release phase extends over between 17 to 27% of the cycle duration and increases the pressure from the pressure at the end of the vacuum maintenance phase to the pressure at the start of the vacuum start phase.

2. The suction profile according to claim 1, wherein the vacuum start phase includes a first subphase and a second subphase following the first subphase, wherein the first subphase extends over between 7% to 13% of the cycle duration and has an average pressure slope of between −15 and −25 mmHg/s and wherein the second subphase extends over between 5 % to 15% of the cycle duration and has an average pressure slope of between −195 and −250 mmHg/s.

3. The suction profile according to claim 2, wherein the first subphase extends over 10% +/−2% of the cycle duration and has an average pressure slope of-20 mmHg/s +/−2 mmHg/s and wherein the second subphase extends over 10% +/−2 % of the cycle duration and has an average pressure slope of-200 mmHg/s +/−10.

4. The suction profile according to claim 1, wherein the vacuum start phase extends over 18% +/−2 % of the cycle duration and has an average pressure slope of −100 mmHg/s +/−10 mmHg/s.

5. The suction profile according to claim 1, wherein the vacuum application phase extends over 30% +/−3 % of the cycle duration and has an average pressure slope of −600 mmHg/s +/−40 mmHg/s.

6. The suction profile according to claim 1, wherein the vacuum maintenance phase extends over 28% +/−2 % of the cycle duration and has an average pressure slope of −3 mmHg/s +/−1 mmHg/s.

7. The suction profile according to claim 1, wherein the vacuum release phase extends over 22% +/−2% of the cycle duration.

8. The suction profile according to claim 1, wherein the vacuum release phase comprises a maximum slope of between 600 and 1000 mmHg/s.

9. The suction profile according to claim 1, wherein in the vacuum application phase the pressure is reduced in steps.

10. The suction profile according to claim 9, wherein each step of the vacuum application phase is between −18 and −25 mmHg.

11. The suction profile according to claim 9, wherein each step of the vacuum application phase is between −20 mmHg +/−1 mmHg.

12. A breast pump including a breast shield adapted to at least in part receive a breast of a lactating user, an aggregate for generating a cyclic suction profile for expressing milk from the breast, a reservoir for the expressed milk, a controller for controlling the aggregate, wherein the controller is adapted to control the aggregate to provide at least one cyclic suction profile according to claim 1.
